(19) United States
(12) Patent Application Publication
(10) Pub. No.: US 2008/0119729 A1
(43) Pub. Date: May 22, 2008

(54) Built-In Balloon Actuator for Urological Device

(76) Inventors: Vincent G. Copa, Minnetonka, MN (US); Justin M. Crank, Maple Grove, MN (US)

Correspondence Address: AMS RESEARCH CORPORATION, 10700 BREN ROAD WEST, MINNETONKA, MN, 55343, US

(21) Appl. No.: 11/943,930

(22) Filed: Nov. 21, 2007

Related U.S. Application Data
(60) Provisional application No. 60/866,878, filed on Nov. 22, 2006.

(51) Int. Cl. A61B 6/00; A61M 29/02; A61M 31/00

(52) U.S. Cl. 600/435; 604/101.01; 604/517

(57) ABSTRACT

An improved catheter design for use in visualizing the prostate. The catheter includes an imaging balloon and a bladder retention balloon. The imaging balloon is disposed along the catheter proximate the location of the prostate. An actuator mechanism is operably connected to the imaging balloon so that the operator can selective inflate/deflate the imaging balloon to correspond with treatment or an ultrasound procedure. The actuator mechanism will allow for various inflation and deflation settings.

### RELATED APPLICATION

The present application claims the benefit of U.S. Provisional Application No. 60/866,878 filed Nov. 22, 2006, which is incorporated herein in its entirety by reference.

### FIELD OF THE INVENTION

The invention relates to devices used in examining and treating prostate problems and more specifically to a device for a urinary catheter used in conjunction with a transrectal ultrasound.

### BACKGROUND OF THE INVENTION

The prostate is a male accessory sex organ located inferior to the urinary bladder and anterior to the rectum. The prostate surrounds and/or encircles the urethra, the tube that connects to the urinary bladder. The prostate is the subject of a number of common disorders including cancer and Benign Prostatic Hyperplasia (BPH). Removal of the prostate can lead to a number of complications including urinary incontinence and erectile dysfunction. Therefore a number of treatments have been developed to treat prostatic conditions without removal.

One such technique for treating cancer of the prostate is known as brachytherapy that involves the transperineal delivery of radioactive implants or seeds, into the stoma of the prostate and in close proximity to the cancerous tissue. The treatment of BPH includes a method known as cryoablation wherein one or more cryoprobes and temperature sensing probes are introduced into the prostate in close proximity with the tissue at issue. Through the cryoprobes, a cold temperature is initiated at the treatment site to create ice balls in the tumor or prostate. Repeated freeze/thaw cycles are performed to cure the malignancy or correct the tissue problem. Another treatment for BPH involves chemical ablation. In one chemical ablation technique, absolute ethanol is injected transurethrally into the prostate tissue. This technique is known as transurethral ethanol ablation of the prostate (TEAP). The injected ethanol causes cells of the prostate to burst, killing the cells. The prostate shrinks as the necrosed cells are absorbed.

The common requirement for both procedures is a detailed knowledge of the patient's anatomical structure. Specifically, for brachytherapy the operator must properly place the radioactive seeds to ensure a successful treatment and limit side effects. Likewise, placing the cryoprobes or injecting the ethanol requires specific placement so as not to damage healthy tissue.

Typically imaging of the prostate is accomplished using transrectal ultrasonography where an ultrasound probe is inserted into the rectum and ultrasound is directed toward the prostate. However, the prostate has a donut shape around the urethra thus preventing the physician from proper imaging of the area. One method of overcoming this deficiency in imaging is by placing a Foley catheter within the urethra proximate the prostate. U.S. Pat. No. 6,863,654 to Zappala et al., incorporated by reference herein, describes a treatment method that fills an imaging bladder by a syringe. Unfortunately, the syringe method of filling the standard catheter does not allow for repeated filling and unfilling contemporaneously with ultrasound operation. Thus there is a need for a catheter that provides greater control during transrectal ultrasonography (TRUS) and prostatic treatments.

### SUMMARY OF THE INVENTION

The present invention is an improved catheter design that provides means for more easily actuating balloons on a urological catheter by incorporating into the hand held portion of a device a fluid reservoir and driving mechanism for inflating/deflating a balloon. The present invention provides an advantage to the TRUS operator in that the urological catheter is easier to fill than with the standard syringe, the typical inflation mechanism. Generally, the present invention contains some number of built-in balloon actuators in the manifold. The actuators perform like a syringe to displace a liquid or gas for selectively inflating and deflating the balloon.

In a first embodiment, the actuator device includes an inflation plunger with locking ribs disposed about the shaft. The locking ribs interact with the body of the plunger to provide more definite locking points. The ribs on the plunger are fixed with a predetermined spacing that corresponds to specific balloon characteristics. The ribs are constructed of a resilient material so that they compress during the axial movement through the plunger aperture yet maintain sufficient stiffness to maintain position in light of the fluid pressure in the plunger.

In an alternate embodiment, the actuator device includes a slider mechanism for controlling the plunger. The slider mechanism is a thumb control style switch mounted external to the plunger body. The thumb switch is operably connected to a plunger for inflating and deflating the balloon. The thumb switch extends through a slot within the plunger. A tab lock section is disposed on each side of the slot to operably interact with a locking tab on the thumb switch. The tab lock section may be configured as a serrated ridge so that each serration provides a locking point for the thumb switch. The thumb switch includes locking tabs extending distally to interact with the tab lock section.

In an alternate embodiment, the actuator device includes a dual plunger controlled by a compressed air fitting. The plunger body includes two sections; a balloon filling section operably connected to a balloon by a lumen, and the control section operably controlled by a spring loaded pin. The operator allows compressed air in to the control section by depressing the spring loaded pin. The compressed air fills the control section forcing the proximal plunger head axially toward the balloon filling section. The distal plunger head forces fluid or gas into the balloon. Further depression of the spring loaded pin allows venting of the compressed air allowing the plunger to retract. As a result the balloon deflates.

Other variations to the present invention are within the scope of the present disclosure. Control of the actuator may be removed from the actual device through the use of a foot pedal which for instance could be used to drive the plunger in any of the embodiment described above. Likewise, a motor may be utilized to drive the plunger arrangement outlined herein.

### BRIEF DESCRIPTION OF THE DRAWINGS

The invention may be more completely understood in consideration of the following detailed description of various embodiments of the invention in connection with the accompanying drawings, in which:

FIG. 1 is a schematic illustration of the male anatomy depicting the urethral system and the components of a TRUS system operatively disposed within the patient.

FIG. 2 is a cross sectional view of a first embodiment of the hand held portion of a uretheral catheter including the present invention.

FIG. 3 is a top view of the plunger of the inflation mechanism of the first embodiment of the present invention.

FIG. 4 is an end view of the inflation mechanism of the first embodiment of the present invention.

FIG. 5 is a cross sectional view of a second embodiment of the hand held portion of a uretheral catheter including the present invention.

FIG. 6 is a top view of the slider assembly of the inflation mechanism of the second embodiment of the present invention.

FIG. 7 is a cross sectional view of the third embodiment of the hand held portion of a uretheral catheter including the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF THE DRAWINGS

In the following detailed description of the present invention, numerous specific details are set forth in order to provide a thorough understanding of the present invention. However, it will be obvious to one skilled in the art that the present invention may be practiced without these specific details. In other instances, well-known methods, procedures, and components have not been described in detail so as to not unnecessarily obscure aspects of the present invention.

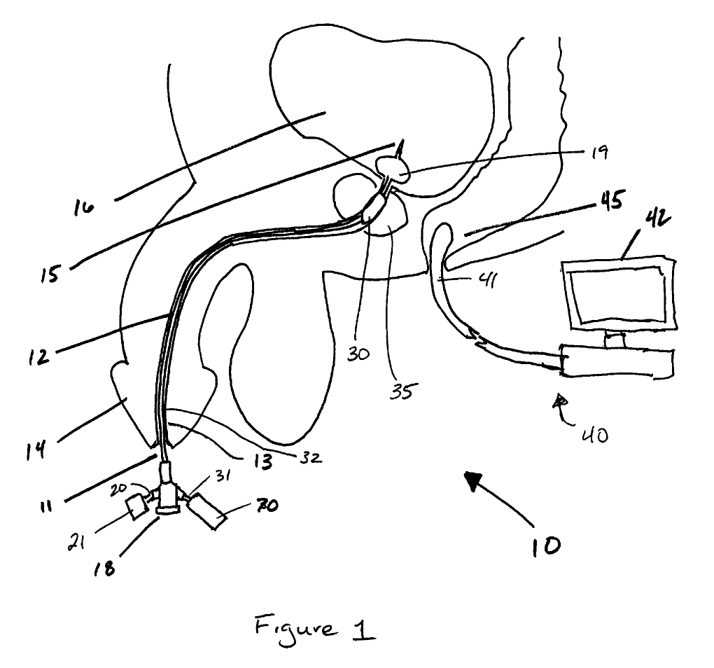

As illustrated in FIG. 1, the prostate treatment system 10 includes a urethral catheter 11. In particular, the urethral catheter 11 includes an elongate catheter 12, similar in length to that of a traditional use Foley catheter. The elongate catheter 12 is structured to be introduced into the urethra 13 of a patient through the penis 14 until a proximate tip 15 thereof extends into a urinary bladder 16 of the patient so as to drain urine from the urinary bladder 16. Furthermore, the elongate catheter 12 also includes a primary lumen 17 that extends generally from the tip 15 that is inserted into the patient's urinary bladder 16 to an exterior, open end 18. In this manner, and through this primary lumen 17 urine may be drained from the patient's urinary bladder 16 during the performance of this and other procedures as necessary, and/or after the procedure.

Further provided in a preferred embodiment of the urethral catheter 11 is a tip balloon or bladder 19. In particular, the tip bladder 19 is formed of a flexible balloon type material and is structured to be effectively expanded upon the introduction of a fluid therein. In order to aid the inflation of the tip bladder 19, a secondary lumen 20 is provided in fluid flow communication between the tip bladder 19 and an inlet port 21. The inlet port 21 may include any desirable valve construction so as to effectively allow for the introduction of a fluid while regulating the escape of a fluid.

In use, the elongate catheter 12 is introduced into the patient's urethra 13 until the tip bladder 19 extends into the urinary bladder 16 of the patient. Once inserted into the urinary bladder 16 of the patient, the tip bladder 19 may thereafter be effectively inflated through the secondary lumen 20. By inflating the tip bladder 19, and as illustrated in the figure, the urethral catheter 12 is essentially maintained in its operative and fluid flow connection with the urinary bladder 16 of the patient. Specifically, the larger size of the tip bladder 19 relative to the opening to the urethra 13 from the urinary bladder 16 is such that removal of the catheter 12 is generally resisted. Furthermore, it noted that although the tip bladder 19 is not a prerequisite for the urethral catheter 11 and prostate treatment system 10 of the present invention, it may be preferred as it will provide a precise positioning of the urethral catheter 12 within the patient. For example, once the tip bladder 19 is inflated, the catheter 12 can be carefully pulled out from the urethra 13 until the tip bladder 19 engages the urinary bladder wall. As a result, a base of the tip bladder 19 will always be disposed at the entrance way to the urethra 13 from the urinary bladder 16.

Looking further to the preferred urethral catheter 12, it also preferably includes an imaging balloon or bladder 30. In particular, the imaging bladder 30 is at least partially, and preferably completely, disposed about an exterior surface of the catheter, although it is recognized that internal placement with appropriate open or flexible construction of the catheter wall can also be achieved. Further, the imaging bladder 30 may be completely cylindrical, helical, fluted, cone shaped or another symmetrical or non-symmetrical shape. A further auxiliary lumen or inflation conduit 32 is also provided and is communicatively disposed between the imaging bladder 30 and an inlet port 31 that includes an actuator. As a result, in use, a fluid may be passed through this auxiliary lumen 31 into inflating position within the imaging bladder 30.

Looking to the preferred embodiments of the imaging bladder 30, it is preferably structured to be a low pressure bladder inflated by a fluid and preferably air, for reasons to be subsequently described. Furthermore, the imaging bladder 30 is preferably formed of a flexible material which may be made of latex or be latex-free material such as including silicone, polyurethane, polyethyleneteraphalete or another latex-free material, so as to allow for appropriate inflation thereof. The preferred material construction of the imaging bladder 30 is achieved so as to minimize the potential obstruction to be generated by the imaging bladder 30 to an imaging device 40, to be described in greater detail subsequently. Furthermore, to aide and/or minimize the obstruction of the imaging, to allow maximum conformance of the imaging bladder 30 to the urethral wall, if desired, and to provide a clearly visible indicator, the imaging bladder 30 will preferably be formed of a substantially thin wall thickness in the range of 0.0001 inches to 0.1 inches, and in the preferred, illustrated embodiments a wall thickness of between 0.001 to 0.005 inches. Further an inflated diameter of approximately 14 Fr (French)-30 Fr may be preferred, with a non-inflated dimension of between approximately 14 Fr-22 Fr may also be desirable. Specifically, and as will be described in greater detail subsequently, the imaging bladder 30 is structured to be inflated under low pressure only until it engages, at least partially, and exerts a mild outward pressure on the urethral wall. As a result, a thick wall, high volume/high pressure structure of the imaging bladder 30 is not required, and indeed in some embodiments may actually be detrimental due to its imaging obstruction. Furthermore, a thin wall thickness and flexible material provides a greater degree of conformity with the urethral wall, if so desired, so that a more accurate image is defined. Moreover, because the practitioner has substantial control over the inflation and/or deflation of the imaging bladder 30 in an on demand type system, the practitioner has substantial control over the viewing process as well, essentially being able to turn on optimized, continuous and manageable imaging of the urethral course, as needed, and until no longer needed.

As can be seen from the Figures, the imaging bladder 30 when operatively disposed with the urethral catheter 12 in the patients urethra 13 is preferably aligned with at least a portion and in many embodiments all of the prostate 35. Specifically, the prostate 35 which is the walnut sized sex organ that wraps around an upper portion of the urethra 13 substantially near the urinary bladder 16 typically has a somewhat standard range of dimensions, at least with regard to the length of the urethra 13 overlapped thereby. Moreover, through various imaging techniques a general determination of the length of the prostate 35 may be determined to select an appropriate sized imaging bladder 30. As a result, the imaging bladder 30 preferably extends through a substantial portion of the urethra 13 that is encased by the prostate 35, and, in the preferred embodiment the imaging bladder 30 is preferably about 4 cm in length. Of course, it is understood that varying lengths may also be provided if greater precision and/or larger coverage area is desired.

Also, the imaging bladder 30 is preferably, although not necessarily, disposed a slightly spaced apart distance from the tip bladder 19 in order to be appropriately positioned relative to the prostate 35. In the illustrated embodiment, the imaging bladder 30 may be closely spaced from the base of the tip bladder 19, that spacing generally positioning the imaging bladder 30 in an appropriately aligned position relative to the prostate 35 when the tip bladder 19 has been inflated and is engaging the walls of the urinary bladder 16.

Further provided as part of the prostate treatment system 10 of the present invention is an imaging device 40. Although the imaging device 40 may include any of a number of different types of imaging devices which provide an accurate, real time view of internal organs, including yet to be developed imaging devices, in the preferred, illustrated embodiments the imaging device 40 includes an ultrasound type system. In this regard, an imaging probe 41 is preferably provided and is structured to emit sound waves in a conventional fashion towards the prostate so as to generate ultrasound images on an associated monitor 42 and processor assembly. In use, the imaging probe 41 is preferably inserted into the rectum 45 of the patient as that provides a substantially close proximity to the prostate 35, and as a result, to the imaging bladder 30 that is located within the prostate 35.

As previously recited, the imaging bladder 30 is preferably inflated with a fluid, and preferably air, through a proximately integrated inflation/deflation device 70. With the imaging bladder 30 generally inflated such that it at least partially and preferably substantially contacts, conforms to and engages the urethral wall, an effective image can be achieved by the imaging device 40. In particular, it is noted that although the urethra 13 is generally not visible and/or readily discernable within the prostate 35 utilizing ultrasound and/or other standard imaging techniques, by inflating the imaging bladder 30 with air, an acoustic interface that is clearly visible utilizing the imaging device 40 is generated and defined. Specifically, the contrast between the fluid disposed within the imaging bladder 30 and the urethral wall defines the acoustic interface, thus allowing a practitioner utilizing the image device 40 to readily view, on their monitor 42, a boundary of the urethra as the contrast point. This boundary of the urethra 13 may then be monitored during performance of a necessary procedure, such as the effective location of a treatment element 80 in the prostate 35. In this regard, it is also noted that the fluid utilized to inflate the imaging bladder may include a radio-opaque material or other contrast medium that can be viewed using ancillary imaging modalities including fluoroscopy as the imaging device 40, and/or if desired; the imaging bladder may be pre-inflated partially and/or completely.

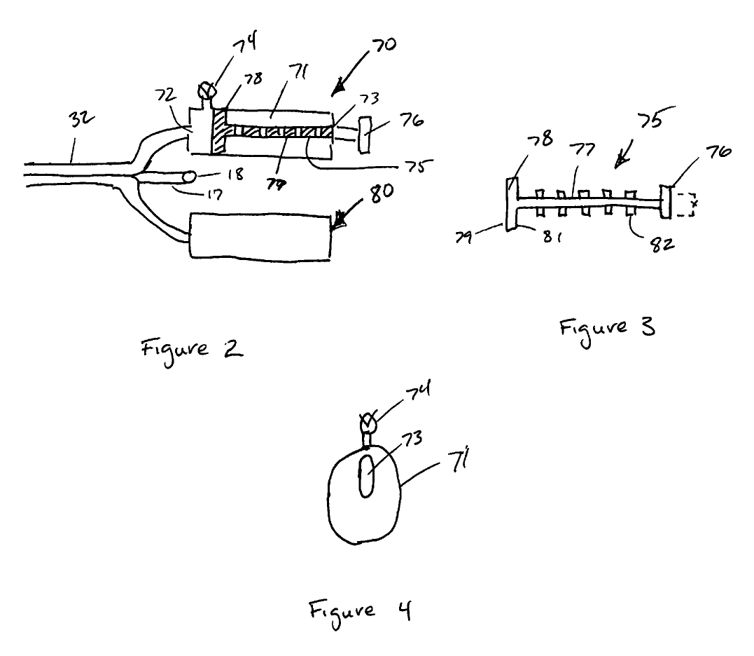

As illustrated in FIGS. 2-4, the first embodiment of the imaging bladder actuator 70 is disclosed along with the treatment system 80. Actuator 70 includes a chamber 71 with a lumen aperture 72 at a first end and a plunger aperture 73 at an opposing end. A check valve 74 is disposed proximate the lumen aperture 72 for controlling the load within the chamber 71. A plunger 75 extends axially within chamber 71. The plunger 75 includes handle 76 connected by shaft 77 to head 78. The handle 76 extends distally from chamber 71 through plunger aperture 73. The handle 76 in a first embodiment is a disk extending generally transverse to shaft 77. The handle 76 has a diameter greater than the diameter of the plunger aperture 73. Head 78 is a disk shaped structure sized to closely fit within the inner perimeter of chamber 71. Head 78 may include a sealing gasket about its outer perimeter to effectuate a seal between the front head face 79 and the rear head face 81.

Shaft 77 includes a series of ribs 82 mounted on opposing sides of the shaft 77. The ribs 82 extend distally from shaft 77. Each rib 82 has a matching rib 82 disposed on the opposite side of the shaft 77. The distance “x” from distal margin of rib 82 to the distal margin of opposing rib 82 is greater than the width of plunger aperture 73. The ribs 82 are constructed of a resilient material capable of bending or compressing when forced through plunger aperture 73. In operation, therefore, the operator must apply sufficient force on handle 76 to direct shaft 77 through plunger aperture 73. The ribs 82 will abut the interior of chamber 71 due to the pressure within the chamber 71 on the front head face 79.

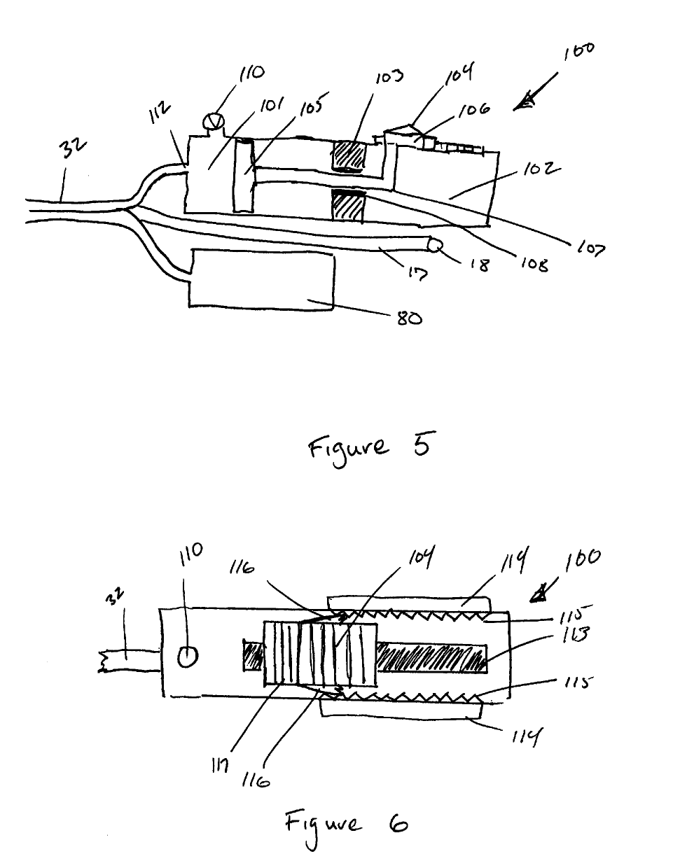

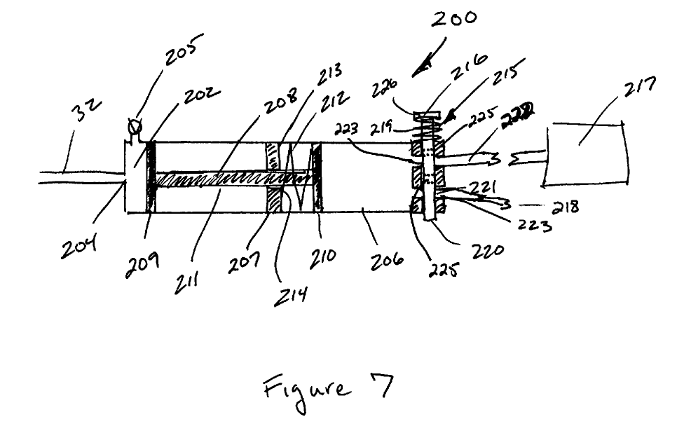

As illustrated in FIG. 5-6, an alternate embodiment actuator 100 may use a thumb control to inflate and deflate the imaging balloon 30. Actuator 100 includes a pressure chamber 101 and a switch chamber 102 separated by seal 103 and a thumb control system 104. The pressure chamber 101 is operably connected to imaging balloon 30 by lumen 32 through lumen aperture 112. The thumb control system 104 includes a plunger 105 disposed within pressure chamber 101. The plunger 105 is operably connected to the thumb switch 106 by plunger arm 107. The plunger arm 107 mounts at a first end to the rear face of plunger 105. The plunger arm 107 extends through seal aperture 108 to connect to thumb switch 106. A check valve 110 is operably connected to pressure chamber 101.

Thumb control system 104 includes thumb switch 106 slidingly engaged to the upper surface of switch chamber 102 within slot 113. On each side of slot 113 are ratchet ramps 114 that include notches 115 for thumb switch 106 adjustments. The thumb switch 106 includes locking tabs 116 that extend transverse to slot 113 for engagement with the ratchet ramps 114. Thumb switch 106 has a triangular shape wherein the base includes the point of attachment for plunger arm 107 and the oblique faces are positioned for operator contact. The oblique faces may contain traction grooves 117.

In operation, the operator advances thumb switch 106 within slot 113 toward the pressure chamber 101 to inflate the imaging balloon 30. The thumb switch 106 is reversed to deflate the imaging balloon 30. The interaction between the locking tabs 116 and the ratchet ramps 114 restrict the motion of thumb switch 106 so that position is maintained when the balloon 30 is at desired inflation point.

FIG. 7 illustrates an alternate embodiment of actuator 200. An imaging balloon 30 is operably connected by lumen 32 to pressure chamber 202 through lumen aperture 204. A check valve 205 is operably connected to the pressure chamber 202 to control flow. The pressure chamber 202 is separated from expansion chamber 206 by seal 207. A two headed plunger 208 extends through the seal 207. Plunger 208 includes a balloon side head 209 and an expansion chamber head 210 connected by plunger shaft 211. A spring 212 is mounted to the rear face 213 of expansion chamber 206 and extends to the seal 207. Both balloon side head 209 and expansion chamber head 210 are closely dimensioned to the interior dimension of pressure chamber 202 and expansion chamber 206, respectively, so that a seal exists about the periphery. It is envisioned that the balloon side head 209 and the expansion side head 209 include a gasket about the respective perimeter to better interact in a sealed manner with the interior walls. Likewise, plunger shaft 211 is closely sized to fit through seal aperture 214 so that the fluid or gas for the imaging balloon 30 does not migrate into the expansion chamber 206 and so that compressed air from the expansion chamber 206 does not migrate into the pressure chamber 202.

Expansion chamber 206 is filled through a valve system 215 disposed at a posterior end of the expansion chamber 206. The valve system 215 includes a spring loaded pin valve 216 operably connected to a compressed gas source 217 and a vent line 218. The compressed gas source 217 may be compressed air or a similar gas. The spring loaded pin valve 216 includes a finger button 226 with a spring 219 attached to its underside that surrounds pin 220. The spring 219 terminates at the exterior of the actuator 200 while pin 220 extends into the expansion chamber 206. Pin 220 resides within pin channel 221 within expansion chamber 206. The pin 220 includes at least two pin openings 225 that correspond with a matching channel opening 223 in the pin channel 221. The compressed gas line 222 is connected from compressed source 217 to channel opening 223. Likewise, a vent line 218 is operably connected to a channel opening 223. The pin openings 225 in pin 220 are positioned so that only one pin opening 225 is aligned with a channel opening 223 at a time so that the expansion chamber 206 cannot be filling and venting simultaneously.

In operation, the operator depressed finger button 226 to a first position that aligns pin opening 225 with channel opening 223 for operable connection with compressed gas source 217. As expansion chamber 206 fills with compressed gas the plunger 208 is forced into pressure chamber 202 forcing gas or liquid into imaging balloon 30. Further depression of finger button 226 to a second position aligns pin opening 225 with channel opening 223 for operable connection with vent line 218 while simultaneously closing the connection to compressed gas source 217. The compressed gas is allowed to vent thus allowing plunger 208 to retract. In the alternative, the compressed gas source 217 could be operably commented to expansion chamber 206 posterior and anterior to expansion side head 209 so that the compressed gas could be used to push the plunger 208 to a fill and empty position for the imaging balloon 30.

Utilizing, the preceding prostate treatment system 10 and the urethral catheter 12, it is further seen that the present invention may be directed towards a method of identifying a patient's urethral anatomic course, in real time, for the precise placement of a treatment element 80 into the patient's prostate 35. In use, the present method may include an initial step of introducing a catheter that has at least an external imaging bladder 30, and in some preferred embodiments a tip bladder 19 into the urethra 13 of the patient until the image bladder 30 is generally aligned with a treatment site of the prostate 35, and in some embodiments until the tip bladder 19 is disposed within the urinary bladder 16. When appropriate, the tip bladder 19 may be effectively inflated thereby securing the catheter within the urethra 13 of the patient. Furthermore, also when appropriate, the imaging bladder 30 is preferably inflated, preferably utilizing a fluid such as air, and preferably until the exterior wall of the imaging bladder 30 generally abuts and/or engages at least a portion of the urethral wall at the prostate 35. In this regard, it may be preferred that the imaging bladder 30, which as previously recited may have a substantially thin wall thickness, will generally conform to the anatomic course of the urethra 13 and will only exert a mild pressure on the urethra 13, although minimal contact is also possible.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

1. An apparatus for placement in the urethra including:
an elongated tubular member having a proximal end portion and a distal end portion, said distal end portion for insertion through the urethra into the bladder of a patient, a fluid passage extending from the proximal end to a drainage portion disposed proximate the distal end portion;
a first balloon disposed proximate the distal end portion, said first balloon operably connected to an inflation lumen extending from said first balloon through the tubular member to the proximal end portion; and
a second balloon attached to the tubular member and operably connected to a second inflation lumen, the second inflation lumen attached to an inflation manifold having an actuator device for selectively inflating and deflating the second balloon, the actuator device including a plurality of set points for controlling the volume of material flowing to the second balloon.

2. The apparatus of claim 1, wherein the actuator device is an inflation plunger with a plurality of locking ribs disposed about the plunger shaft, said locking ribs being oversized with respect to a plunger aperture in the actuator device so as to define set-points for inflating and deflating the second balloon.

3. The apparatus of claim 2, wherein the locking ribs are constructed of a resilient material so as to flex sufficiently during advancement of the locking ribs through the plunger aperture without snapping.

4. The apparatus of claim 1, wherein the actuator device includes a slider mechanism operably connected to an inflation plunger, the slider mechanism engaging a slot in the actuator device so as to selectively advance or retract the inflation plunger with the slider mechanism.

5. The apparatus of claim 4, wherein the slider mechanism comprises a thumb switch, the thumb switch attached to a plunger arm having a first portion extending through the slot in the actuation device and a second portion attached to the inflation plunger.

6. The apparatus of claim 5, wherein the thumb switch includes at least one locking tab that slidably engages a ratcheting section disposed about the slot.

7. The apparatus of claim 6, wherein the ratcheting section includes ratchet ramps along each side of the slot, the ratchet ramps defining a plurality of individual notches for engaging the at least one locking tab.

8. The apparatus of claim 1 wherein the actuation device includes a compressed gas system, an inflation plunger and a valve system, the inflation plunger including a first head and a second head connected by a plunger shaft, wherein the compressed gas system and valve system provide a pressurized gas to bias the second head such the first head controls a volume of material directed to the second balloon.

9. The apparatus of claim 8, the valve system includes a spring loaded pin valve and a pin channel operably interfacing with the compressed gas source.

10. The apparatus of claim 9, wherein the spring loaded pin valve includes a finger control portion.

11. The apparatus of claim 9, wherein the spring loaded pin valve includes a fill opening and a vent opening and said pin channel including a fill access channel and a vent access channel, the spring loaded pin valve being actuatable to selectively align the fill opening with the fill access channel to inflate the second balloon and to selectively align the vent opening with the vent access channel to deflate the second balloon.

12. The apparatus of claim 11, wherein the fill opening and the vent opening on the spring loaded pin valve are arranged so as to avoid simultaneous alignment with the fill access channel and the vent access channel.

13. A method for treating the prostate including:
providing a catheter having an imaging balloon;
advancing the catheter into a urethra of a patient such that the imaging balloon is aligned within a portion of the urethra within a prostate;
inflating the imaging balloon with an actuator device to a desired inflation size so as to selectively define an acoustic interface between the urethra and the prostate for visualization with an ultrasound imaging device.

14. The method of claim 13, further comprising:
inflating a first balloon on a tip portion of catheter in a bladder of the patient.

15. The method of claim 13, wherein inflating the imaging balloon with the actuator device comprises advancing a plunger member having a plurality of ribs past a plunger aperture to selectively provide a desired amount of inflation fluid to the imaging balloon.

16. The method of claim 13, wherein inflating the imaging balloon with the actuator device comprises advancing a thumb control along a ratcheted slot to selectively provide a desired amount of inflation fluid to the imaging balloon.

17. The method of claim 13, wherein inflating the imaging balloon with the actuator device comprises advancing a plunger member having a first head and a second head, wherein a compressed gas source selectively biases the second head to advance or retract the plunger member such that the first head provides a desired amount of inflation fluid to the imaging balloon.

18. An imaging system for viewing the prostate comprising:
an elongated tubular member having a proximal end portion and a distal end portion, said distal end portion for insertion through the urethra into the bladder of a patient, a fluid passage extending from the proximal end to a drainage portion disposed proximate the distal end portion;
a first balloon disposed proximate the distal end portion, said first balloon operably connected to an inflation lumen extending from said first balloon through the tubular member to the proximal end portion;
a second balloon attached to the tubular member and operably connected to a second inflation lumen, the second inflation lumen attached to an inflation manifold having an actuator device for selectively inflating and deflating the second balloon, the actuator device including a plurality of set points for controlling the volume of material flowing to the second balloon; and
an imaging system for identifying an acoustical interface between the filled second balloon and the prostate.

19. The imaging system of claim 18, wherein the imaging system comprises an ultrasound imaging system.

20. The imaging system of claim 18, wherein the material flowing to the second balloon is a fluid selected from the group consisting of: air and a radio-opaque material.

21. The apparatus of claim 19 wherein the inflation means includes an imaging bladder actuator control, operably connected to a plunger within an inflation chamber, said imaging bladder actuator control having multiple inflation volume settings.
